(19) United States
(12) Patent Application Publication
(10) Pub. No.: US 2004/0059422 A1
(43) Pub. Date: Mar. 25, 2004

(54) Implant for cranioplastics

(76) Inventors: Karl Koschatzky, Erlangen (DE); Matthias Lowel, Nurnberg (DE)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/239,619

(22) PCT Filed: Jan. 29, 2001
(86) PCT No.: PCT/EP01/00936

(30) Foreign Application Priority Data
Mar. 24, 2000 (DE) 100 14 616.3

(51) Int. Cl. A61F 2/02

(52) U.S. Cl. 623/17.19

(57) ABSTRACT

The invention relates to a cranium implant that consists of an essentially plate-shaped implant body (1) made of spongious, cortical or compact bone material of human or animal origin.

The present invention relates to an implant for cranioplasty.

In the course of operations on the brain, the cranium has to be opened (cranioectomy). After the operation, but optionally also after a certain time interval after the operation (e.g. after the cessation of cerebral edema), the defect created in the cranium must be closed, Either polymer-based bone cements or metal plastics, e.g. made of gold or titanium, are used here.

It is disadvantageous here that, with polymers, in addition to the foreign body reaction, there is a risk of thermal damage to the patient's tissue by exothermal reactions on the hardening of the bone cements. Moreover, unwanted local and/or systemic side effects can be caused by monomers or oligomers being released.

Metallic implants remain in the implant bed for life and result in artifact formations in the imaging in diagnostic exminations such as X-rays. A diagnosis of the healing success after the operation is thus only possible with limitations. Moreover, with metallic implants the processing and the matching to the operation wound is as good as impossible as part of the primary operation; they have to be implanted in the course of a second operation.

The object of the present invention consists in providing a mechanically stable implant for cranioplasty which does not show the above-mentioned disadvantages and which can be fixed sufficiently securely, wherein a matching of the implant to the defect should be possible with the conventional operating theater instruments.

This object is satisfied by a cranium implant having the features of claim 1.

The implant in accordance with the invention eliminates the initially named disadvantages and neither damages the implant bed nor are imaging processes influenced. The implant can be absorbed over a longer period of time and can be replaced by the body's own tissue without causing damage to the implant bed by absorption products.

Advantageous embodiments of the invention are described in the dependent claims.

The implant can be formed as follows in accordance with the cranioectomy:

polygonal, circular, ellipsoid,

planar, vaulted.

The implant can have fixation aids such as drillings to fix the implant to the body's own cranial bone.

A particular advantage of the implant in accordance with the invention is provided by the material used which, due to its biological origin, does not represent a foreign body. The implant manufactured from bone material is converted into the body's own tissue during incorporation.

An application in other surgical measures such as the fixation and plating of bone fragments after fractures or bodies of a vertebra is possible, in addition to an application on the cranium, due to the differently sized dimensions and designs of the implant.

Further advantageous embodiments of the cranium implant in accordance with the present invention will be specified in the description, in the drawing and in the dependent claims.

In accordance with a preferred embodiment of the invention, the material of the implant consists of conserved and sterile bone material of human or animal origin, in particular of conserved and sterile bone material of bovine, porcine or equine origin.

The bone material can consist of spongious, cortical, but preferably compact, bone, or of composites resulting therefrom.

A suitable allogenic or xenogenic bone material is processed as the material for the implant in accordance with the present invention such that it is conserved, storable and sterile and can be used for the intended purpose. The conservation of the bone material can take place, for example, by means of freeze drying. However, the bone material is preferably produced by solvent dehydration of bone material containing collagen by means of an organic solvent miscible with water, e.g. methanol, ethanol, propanol, isopropanol, acetone, methyl ethyl ketone or mixtures of these solvents. The conservation and sterilization of the bone material in accordance with this process is also the subject of the patent DE 29 06 650, whose contents are incorporated by reference into the disclosure of the present application.

This process serves for the manufacture of preserved transplant materials and allows a dehydration and exposure down to the fine structure of the fibril of the collagen bone material such that the processed bone material has a practically identical structure to the natural bone in the histological picture and such that the desired properties of the collagen bone material are thus maintained. This process of solvent dehydration, moreover, has the advantage that a much lower apparatus effort is required in comparison with freeze drying.

Furthermore, the bone material can also be manufactured by solvent dehydration of bone material containing collagen with a subsequent terminal sterilization, in particular by irradiation with gamma rays or electron rays, but also by ethylene oxide or thermal processes.

Alternatively, the bone material can be produced by aseptic processing of collagen bone material without terminal sterilization.

The present invention will be described below purely by way of example with reference to an embodiment of a cranium implant in accordance with the invention and to the enclosed drawings.

The single FIGURE shows a perspective view of an embodiment of a cranium implant in accordance with the present invention.

The embodiment shown in the FIGURE is suitable for the implantation into the cranium and includes an implant body 1 which, in the embodiment shown, consists of compact bone material of bovine origin, The implant body 1 has a plurality of passage drillings 2 which serve for the fixation of the implant to the cranium.

The implant body 1 is made substantially rectangular in a plan view and has a slight vaulting 3.

The implant body 1 of the cranium implant is generally matched to the implant bed in its dimensions. The outer dimensions of such a cranium implant can be as follows, for example, depending on the application site: length: 5 to 150 mm; width: 5 to 150 mm; and thickness: 0.5 to 10 mm. The passage drillings 2 are matched to corresponding fixation elements and have a diameter of approximately 0.5-2.5 mm. The angle of the vaulting is matched to the implantation site and ranges from 0° to 180°.

Reference Numeral List

1 implant body

2 passage bore

3 vaulting

1. A cranium implant consisting of a substantially plate-like implant body (1) of spongious, cortical or compact bone material of human or animal origin.

2. A cranium implant in accordance with claim 1, characterized in that the implant body (1) is made vaulted.

3. A cranium implant in accordance with claim 1, characterized in that the material of the implant body (1) consists of conserved and sterile bone material.

4. A cranium implant in accordance with any one of the preceding claims, characterized in that the implant body (1) has one or more passage drillings (2) to fix the cranium implant to the cranium.

5. A cranium implant in accordance with any one of the preceding claims, characterized in that the bone material is produced by solvent dehydration of collagen bone material by means of an organic solvent miscible with water such as methanol, ethanol, propanol, isopropanol, acetone, methyl ethyl ketone or mixtures of these solvents.

6. A cranium implant in accordance with any one of the preceding claims, characterized in that the bone material is produced by solvent dehydration of collagen bone material with subsequent terminal sterilization, in particular by irradiation with gamma rays or electron rays.

7. A cranium implant in accordance with any one of the preceding claims 1 to 5, characterized in that the bone material is produced by aseptic processing of collagen bone material without terminal sterilization.
